(19) United States
(12) Patent Application Publication
(10) Pub. No.: US 2025/0099123 A1
(43) Pub. Date: Mar. 27, 2025

(54) LITHOTRIPSY DEVICE FOR BREAKING UP BODY STONES, LITHOTRIPSY SYSTEM, RETROFIT KIT FOR RETROFITTING AN EXISTING LITHOTRIPSY DEVICE, AND METHOD FOR OPERATING A LITHOTRIPSY DEVICE

(71) Applicant: KARL STORZ SE & Co. KG, Tuttlingen (DE)

(72) Inventor: Bernhard GLÖGGLER, Tuttlingen (DE)

(21) Appl. No.: 18/710,681

(22) PCT Filed: Nov. 29, 2022
(86) PCT No.: PCT/EP2022/083659

§ 371 Date: Nov. 26, 2024

(30) Foreign Application Priority Data
Dec. 1, 2021 (DE) 10 2021 131 669.3

(51) Int. Cl. A61B 17/225

(52) U.S. Cl. CPC A61B 17/225

(57) ABSTRACT

The invention relates to a lithotripsy device for fragmenting body stones, wherein the lithotripsy device has a hand-held device with an ultrasonic vibration exciter with an ultrasonic vibration exciter outer diameter and a sonotrode with a longitudinal central axis and a sonotrode outer diameter which can be connected to the hand-held device on the distal side, wherein the sonotrode can be excited to a first vibration by means of the ultrasonic vibration exciter, and the lithotripsy device has a vibration excitation element with a through-opening, wherein the vibration excitation element with the through-opening is arranged around the sonotrode outer diameter and/or around the ultrasonic vibration exciter outer diameter, wherein the through-opening of the vibration excitation element has a larger diameter than the sonotrode outer diameter and/or than the ultrasonic vibration exciter outer diameter so that the vibration excitation element is freely movable, and the vibration excitation element has an imbalance so that, in the event of a rotation of the vibration excitation element about the sonotrode and/or the ultrasonic vibration exciter, the sonotrode can be excited to a second vibration by means of the imbalance. Furthermore, the invention relates to a lithotripsy system, a retrofitting kit for retrofitting an existing lithotripsy device, and a method for operating a lithotripsy device.

The invention relates to a lithotripsy device for fragmenting body stones, wherein the lithotripsy device has a hand-held device with an ultrasonic vibration exciter with an ultrasonic vibration exciter outer diameter and a sonotrode with a longitudinal central axis and a sonotrode outer diameter which can be connected to the hand-held device on the distal side, wherein the sonotrode can be excited to a first vibration by means of the ultrasonic vibration exciter, and the lithotripsy device has a vibration excitation element with a through-opening, wherein the vibration excitation element with the through-opening is arranged around the sonotrode outer diameter and/or around the ultrasonic vibration exciter outer diameter. Furthermore, the invention relates to a lithotripsy system, a retrofitting kit for retrofitting an existing lithotripsy device, and a method for operating a lithotripsy device.

Lithotripsy is a well-known procedure for fragmenting body stones which form as a so-called concretion in body organs, e.g., in the bladder or kidney, due to the crystallization of salts. If the body stones are too large for natural excretion and cause discomfort, they must be fragmented using a lithotripter so that the fragmented stones can be removed through natural excretion and/or using a suction-irrigation pump.

Such body stones are often not homogeneously structured, but have different components, layers, and/or solidities. A body stone which has a soft concretion on the outside and a hard stone core on the inside therefore requires, with increasing fragmentation, more fragmenting force by the shock waves emitted by the lithotripter.

For these reasons, purely ultrasound-based lithotripters have been further developed in recent years to improve stone fragmentation performance. In addition to the constant ultrasound energy, intermittent ballistic shock wave energy is often applied. This can be done, for example, by means of a ballistic drive with electromagnets, in which an impact body is accelerated by the electromagnets and strikes a horn and/or the sonotrode head. The disadvantage of such ballistic drives is that they must be actively cooled starting at a certain electrical power consumption, since otherwise the surface temperature will be too high.

U.S. Pat. No. 9,421,023 B2 discloses a device for transmitting ultrasonic vibrations in which the ultrasonic waveguide is accommodated proximally in a waveguide fitting, and, distally to the waveguide fitting, a first shock-pulsing mass followed by a compression spring and a second shock-pulsing mass are arranged around the ultrasonic waveguide. The second shock-pulsing mass is bounded on the distal side by an impact surface onto which the second shock-pulsing mass strikes when excited by the ultrasonic vibration. The disadvantage here is that the second shock-pulsing mass is excited only by the high-frequency ultrasonic vibration and is directly dependent upon it. This means that the vibration behavior of the shock-pulsing mass and the shock waves it generates can only be slightly influenced. Due to the arrangement of the compression spring between the two shock-pulsing masses around the outside of the ultrasonic waveguide, the compression spring, as a store of the supplied ultrasonic energy, transmits said energy only along the longitudinal central axis of the ultrasonic waveguide, and the impact of the second shock-pulsing mass on the impact surface always occurs from an axial direction and therefore coaxially to the longitudinal central axis of the ultrasonic waveguide. In addition, this limitation means that the device cannot be retrofitted in ultrasonic transducers and sonotrodes already on the market.

The object of the invention is to improve the state of the art.

The object is achieved by a lithotripsy device for fragmenting body stones, wherein the lithotripsy device has a hand-held device with an ultrasonic vibration exciter with an ultrasonic vibration exciter outer diameter and a sonotrode with a longitudinal central axis and a sonotrode outer diameter which can be connected to the hand-held device on the distal side, wherein the sonotrode can be excited to a first vibration by means of the ultrasonic vibration exciter, and the lithotripsy device has a vibration excitation element with a through-opening, wherein the vibration excitation element with the through-opening is arranged around the sonotrode outer diameter and/or around the ultrasonic vibration exciter outer diameter, wherein the through-opening of the vibration excitation element has a larger diameter than the sonotrode outer diameter and/or than the ultrasonic vibration exciter outer diameter so that the vibration excitation element is freely movable, and the vibration excitation element has an imbalance so that, in the event of a rotation of the vibration excitation element about the sonotrode and/or the ultrasonic vibration exciter, the sonotrode can be excited to a second vibration by means of the imbalance.

This provides a lithotripsy device with a defined dynamic and/or static imbalance of the vibration excitation element, which wobbles around the sonotrode during excitation and/or rotation, and thereby exerts axial and/or radial pulses upon the sonotrode. The imbalance mass as the impact mass of the vibration excitation element and the thereby generated intermittent shock impacts on the sonotrode improve the performance of fragmenting body stones and the drilling capability of the sonotrode, even with hard and/or inhomogeneous body stones. In so doing, the shock pulses of the moving and/or tumbling vibration excitation element are superimposed with the continuous ultrasonic vibrations generated by the ultrasonic vibration exciter so that two mechanisms of vibration excitation and stone fragmentation are active simultaneously or intermittently.

It is particularly advantageous that the vibration excitation element, due to the larger diameter of its opening, which surrounds the sonotrode, a horn, and/or the ultrasonic vibration exciter, compared to the outer diameter of the sonotrode, the horn, and/or the vibration exciter, is freely movable on and around the sonotrode, the horn, and/or the ultrasonic vibration exciter and is not at all fixedly installed in one direction or several directions.

In principle, it should be noted that, even if the arrangement of the vibration excitation elements around the sonotrode is referred to below, this always also means that the vibration excitation element or a second vibration excitation element or several vibration excitation elements can alternatively or in addition also be arranged analogously around the horn and/or the ultrasonic vibration exciter.

Due to the free movement of the vibration excitation element around the sonotrode and due to its imbalance, not only regular, but also irregular shock pulses, in terms of time, location, and/or strength, can be transmitted to the sonotrode. By superimposing the continuous ultrasonic vibration with the irregular shock pulses of the freely moving vibration excitation element on the connected sonotrode, a combined effect and therefore improved body stone removal is achieved, compared to the ultrasound-based vibration excitation of the sonotrode alone. Due to the free movement of the vibration excitation element, the sonotrode also vibrates laterally, thereby reducing the contact time in the borehole of the body stone. This results in less friction and lower energy losses by the ultrasonic transducer.

Due to the mobility of the vibration excitation element in all spatial directions because of the width of the through-opening arranged around the sonotrode, impacts in the direction of the Z-axis and therefore lateral impulse impacts on the sonotrode can also be carried out.

The fact that the vibration excitation element surrounds the sonotrode, wobbles freely around the sonotrode due to the imbalance and causes a direct transfer of energy to the sonotrode by impacting, for example, with its edges arranged around the opening on the outer surface of the sonotrode, directly improves stone fragmentation. In so doing, it is advantageous if the impact surfaces of the sonotrode and/or the vibration excitation element are not deformed, which can be achieved by selecting an appropriate material.

The irregular, intermittent shock pulses generated by the vibration excitation element with the imbalance are also amplified by the fact that the shock pulses are reflected at the tip and/or at the head of the sonotrode.

An essential idea of the invention is based upon the fact that at least one vibration excitation element is arranged to be freely movable around a sonotrode (and/or horn and/or ultrasonic vibration exciter) and is not clamped by means of a compression spring as an energy store, whereby a free, random impact of the vibration excitation element from all spatial directions on the outer surface of the sonotrode is enabled when the vibration excitation element moves and/or rotates around and/or along the sonotrode. The freely movable arrangement of the vibration excitation element around the sonotrode makes it easier to drive it to move and/or rotate, its drive can be adapted more easily, and the vibration excitation element can also be retrofitted to sonotrodes and/or ultrasonic transducers already on the market.

### The Following Terminology is Explained:

A “lithotripsy device” (also known as a “lithotripter”) is in particular a device for fragmenting body stones using shock waves. A lithotripsy device is understood in particular to mean various parts, structural and/or functional components of a lithotripter. The lithotripsy device can completely or partially form a lithotripter. A lithotripsy device can in particular be an intracorporeal or extracorporeal lithotripsy device. In the case of an intracorporeal lithotripsy device, this may also have an irrigation/suction pump. The lithotripsy device can be designed as a hand-held device and/or have an endoscope or be inserted into an endoscope. The lithotripsy device is in particular autoclavable and, for example, made of instrument steel and/or plastic. The lithotripsy device can have further components such as a control and/or supply device, or these are assigned to the lithotripsy device.

The term “body stones” (also known as “concretion”) refers in particular to all stones in a human or animal body that are formed from salts through crystallization. Body stones can, for example, be gallstones, urinary stones, kidney stones, and/or salivary stones.

A “hand-held device” is in particular a handle for manual and/or automated operation and/or a connection of the lithotripsy device. The hand-held device can be designed as a hand-held and/or holding part. The hand-held device can also be arranged, connected, and/or automatically guided on a distal end of a robot arm. In particular, the hand-held device has an attachable holder housing.

An “ultrasonic vibration exciter” (also called a “vibration exciter”) is in particular a component of an ultrasonic transducer and/or handpiece of a lithotripsy device which converts a supplied alternating voltage at a certain frequency into a mechanical vibration frequency. The ultrasonic vibration exciter is in particular an electromechanical transducer utilizing the piezoelectric effect. By applying an alternating electrical voltage generated by an ultrasonic generator, a mechanical vibration is generated in particular due to deformation of the ultrasonic vibration exciter. In particular, the ultrasonic vibration exciter has one or more piezo elements. Preferably, the ultrasonic vibration exciter has at least two piezo elements, wherein an electrical conductor, e.g., a copper disk, can be arranged between the piezo elements. The ultrasonic vibration exciter and/or the ultrasonic transducer can in particular have a horn.

A “horn” is in particular a component that is arranged between the vibration exciter and/or a piezo element and the sonotrode. The horn is used in particular to transmit, forward, and/or align the ultrasonic waves generated by the vibration exciter to the sonotrode. For this purpose, the horn can taper in a transmission direction and transmit the ultrasonic waves directly or indirectly to a sonotrode head. The horn can also be used to attach the sonotrode. At the same time, the horn serves to mechanically hold the piezo element or piezo elements on both sides—in particular, together with a counter bearing.

The “longitudinal central axis” is in particular the axis of the sonotrode that corresponds to the direction of its greatest extension.

A “sonotrode” is in particular a component that itself is set into vibration and/or resonant vibration by the action and/or introduction of mechanical vibrations. In particular, the sonotrode is excited to a first vibration by means of the ultrasonic vibration exciter. In addition, the sonotrode is excited to a second vibration in particular by means of the imbalance of the vibration excitation element. The sonotrode is designed in particular as a waveguide for the shock waves (pulses) of the vibration excitation element with the imbalance and/or the ultrasonic waves generated by the ultrasonic vibration exciter. In particular, the sonotrode is connected to the ultrasonic vibration exciter, the ultrasonic transducer, and/or the horn. For example, the sonotrode is screwed into the distal end of the horn. In particular, the sonotrode has a sonotrode head at its proximal end for receiving, transmitting, and/or focusing ultrasonic waves, and a sonotrode tip at its distal end for directly and/or indirectly striking and/or contacting body stones. In particular, the sonotrode is shaped in such a way that it optimally introduces the shock waves and/or the second vibration and/or the ultrasonic vibration at its distal end into the body, the body region to be treated, and/or directly onto the body stone to be fragmented. In the case of ultrasonic excitation, the sonotrode operates in particular in the ultrasonic range at a frequency range of 20 KHz to 90 kHz-preferably of 20 KHz to 34 kHz. The sonotrode is made of steel, titanium, aluminum, and/or carbon. A sonotrode is, in particular, a probe that is, for example, rod-shaped, tubular, and/or hose-shaped. The sonotrode can be made in one piece or in several pieces. In particular, the sonotrode has a diameter in a range of 0.5 mm to 4.5 mm-especially of 0.8 mm to 3.8 mm. The sonotrode has an outer diameter that is smaller than the opening of the vibration excitation element.

The “outer diameter” is in particular the external diameter and therefore the greatest possible distance between two points on the outer circular line of the sonotrode (“sonotrode outer diameter”), the horn, and/or the ultrasonic vibration exciter (“ultrasonic vibration exciter outer diameter”). In particular, the outer diameter can be the maximum diameter. However, the outer diameter can also be the diameter that is present in the region of the sonotrode, the horn, and/or the ultrasonic vibration exciter that is surrounded by the vibration excitation element.

The term “vibration excitation element” refers in particular to a body, component, and/or assembly which can be slid onto the sonotrode, the horn, the ultrasonic vibration exciter, and/or the ultrasonic transducer. The vibration excitation element has a defined static imbalance and/or a dynamic imbalance—in particular, due to its properties, such as shape, weight distribution, and/or design, and arrangement of its through-opening. For easy drivability for moving and/or rotating the vibration excitation element, its total mass should be kept as low as possible. However, its total mass can also be as high as possible so that the vibration excitation element can deliver correspondingly hard impacts on the sonotrode. The vibration excitation element is made of metal—for example, stainless steel.

An “imbalance” is present in a rotating body in particular if its axis of rotation does not correspond to one of its main axes of inertia. An imbalance is in particular a static imbalance and/or a dynamic imbalance of the vibration excitation element. The imbalance leads in particular to vibrations, wobbling of the vibration excitation element around the sonotrode, and impact of the inner surface around the through-opening and/or the adjacent edges around the through-opening of the vibration excitation element on the outer surface of the sonotrode. This means that the static imbalance and/or the dynamic imbalance of the vibration excitation element is defined and can be used specifically to excite the sonotrode to a second vibration.

A “static imbalance” exists in particular if the axis of rotation of the vibration excitation element does not run through the center of mass of the vibration excitation element. The static imbalance generates circular mechanical vibrations at a right angle to the axis of rotation. A static imbalance can be specifically generated in particular by attaching an imbalance element to only one outer side of the vibration excitation element. In particular, the imbalance is the product of the imbalance mass and the distance from the axis of rotation. The imbalance can be specified in particular in the unit, mm*g.

A “dynamic imbalance” exists in particular if the axis of rotation of the vibration excitation element does not coincide with one of its stable main support axes, but is tilted in the center of mass relative to the main support axes. The dynamic imbalance causes a bending moment on the axis of rotation—in particular, during operation-which causes circular vibrations displaced by 180° at the ends of the axis of rotation. In particular, the rotating vibration excitation element remains at rest, while the axes wobble due to the opposing circular motion. A dynamic imbalance can be caused, for example, by two opposing imbalance elements attached to different sides of the vibration excitation element. The dynamic imbalance therefore causes the vibration excitation element to wobble and/or tilt away from the transverse axis, which is perpendicular to the longitudinal central axis of the sonotrode.

The “first vibration” excited by the ultrasonic vibration exciter and the “second vibration” excited by the imbalance of the vibration exciter element differ in particular in their properties. The first vibration is in particular a regular and/or constant ultrasonic vibration. In particular, the first vibration has a constant wavelength or several constant wavelengths. The second vibration excited by means of the imbalance of the vibration excitation element is in particular discontinuous and/or intermittent shock waves. The second vibration preferably has a greater intensity and/or amplitude. The second vibration can also have a time-varying wavelength. Thus, the first vibration and the second vibration differ in particular in at least one property, such as in their amplitude, wavelength, period, and/or in an excited resonance vibration of the sonotrode.

“Rotation” refers in particular to a rotational movement of the vibration excitation element. The rotation of the vibration excitation element is in particular the rotational movement of the vibration excitation element about an imaginary central axis as the axis of rotation through the through-opening and/or about the longitudinal central axis of the sonotrode. However, the imaginary central axis does not necessarily have to be arranged transversely to the distal outer surface and the proximal outer surface of the vibration excitation element, which are aligned horizontally in the resting state; instead, the through-opening and therefore the axis of rotation can also run diagonally through the vibration excitation element and therefore not parallel to the longitudinal central axis of the sonotrode. A rotation does not necessarily have to be a complete rotation of 360° or several complete rotations; instead, a rotation is understood in particular to be a partial rotation through <360°. Instead of a rotation, however, the vibration excitation element can also move in one of the possible spatial directions.

In a further embodiment of the lithotripsy device, the vibration excitation element is disk-shaped, annular, hollow cylinder-shaped, and/or toroidal.

Depending upon the shape of the vibration excitation element, this can provide an optimum rotational speed and/or optimally defined impact edges and/or surfaces.

With the disc-shaped, annular, and/or toroidal form of the vibration excitation element, by the arrangement of the wall thickness in the direction of the longitudinal central axis of the sonotrode and of the longer outer diameter in the transverse direction to the longitudinal central axis of the sonotrode, a wobbling of the vibration element with a large deflection at the opposing outer regions around the outer diameter can be realized. This means, for example, that the distal edge and the proximal edge alternately hit the outer surface of the sonotrode around the through-opening.

In order to set a defined dynamic imbalance, the vibration excitation element has an axis of rotation deviating from the longitudinal central axis and/or its main axis of inertia.

Thus, the axis of rotation of the vibration excitation element runs in the direction of the longitudinal central axis of the sonotrode, for example, from a point on the proximal side of the vibration excitation element at a smaller distance from the longitudinal central axis at an angle to a point on the distal side of the vibration excitation element at a greater distance from the longitudinal central axis of the sonotrode.

However, if the axis of rotation of the vibration excitation element deviates from the longitudinal central axis of the sonotrode, a static imbalance can also be generated by the fact that, even though the axis of rotation is perpendicular to the horizontally aligned outer surfaces of the vibration excitation element and therefore parallel to the longitudinal central axis of the sonotrode, it does not coincide with the longitudinal central axis of the sonotrode. This means that the axis of rotation and therefore the through-opening can be shifted outwards along the radius, from the geometric center of the vibration excitation element. Consequently, the axis of rotation runs eccentrically to the longitudinal central axis of the sonotrode.

The “axis of rotation” (also called “rotational axis”) is in particular a straight line that describes a rotation or turning of the vibration excitation element. The axis of rotation of the vibration excitation element is, in particular, the physically real axis of rotation. If the axis of rotation deviates from the longitudinal central axis of the sonotrode, the axis of rotation therefore deviates from the axis of symmetry of the vibration excitation element. This changes the view of the vibration excitation element-particularly when the vibration excitation element is rotated at a random angle.

A “main axis of inertia” (also called “main axis”) refers in particular to an axis of rotation of the vibration excitation element about which the vibration excitation element can be continuously rotated without a dynamic imbalance occurring. The main axis of inertia runs in particular through the center of mass of the vibration excitation element.

In a further embodiment of the lithotripsy device, the vibration excitation element has an imbalance element in its interior and/or on its outer surface.

This means that a static and/or dynamic imbalance can be specifically set by arranging one imbalance element or several imbalance elements on and/or in the vibration excitation element.

An “imbalance element” can, for example, be a mass with a different material and/or weight than the main mass of the vibration excitation element. By selecting the weight of the imbalance element, its size, shape, and/or its arrangement along the radius of the vibration excitation element, an imbalance can be specifically set, and the rotational behavior and the impact behavior of the vibration excitation element can be influenced.

In order to keep the weight of the vibration excitation element low and to configure it to be more easily drivable in a rotational movement but still introduce a targeted imbalance into the vibration excitation element, the vibration excitation element has a recess.

Thus, the planned arrangement of one recess or several recesses in the vibration excitation element can be used to realize an uneven weight distribution along the radius of the vibration excitation element, and therefore a non-rotationally symmetrical weight and/or mass distribution.

A “recess” is in particular a free space in the vibration excitation element which is arranged in its interior and/or on its outer surface. A recess can be a cavity, a depression, and/or an incision in the vibration excitation element. A recess can also be an undercut.

In order to achieve increased wobbling of the vibration excitation element in the distal and/or proximal direction, the imbalance element and/or the recess is and/or are arranged on a distal side and/or a proximal side of the vibration excitation element.

The mass of the imbalance element and the size of the recess as well as its particular arrangement along the radius can therefore be used to adjust the fluctuation width of the wobble and/or the back-and-forth movement in the proximal and distal directions at the opposing regions of the outer diameter of the transverse axis transverse to the through-opening of the vibration excitation element.

The “distal side” is understood to mean in particular the side away from the user and therefore close to the body. Accordingly, the “proximal side” is understood to mean the side close to the user and the side away from the body. In a disk-shaped, annular, hollow cylinder-shaped, and/or toroidal vibration excitation element, the distal and proximal sides are in particular the two opposing circular or annular surfaces.

In order to specifically provide a second drive system for the rotational movement of the vibration excitation element in addition to excitation by the ultrasonic vibration of the ultrasonic vibration exciter, the lithotripsy device has a drive apparatus for driving the rotation of the vibration excitation element.

This allows the desired second vibration of the sonotrode to be set flexibly by means of a second active drive apparatus which is specifically used solely to drive the rotational movement of the vibration excitation element. This second drive apparatus is therefore independent of the excitation by the ultrasonic vibration exciter and is not limited by the structural design of the components of the ultrasonic vibration exciter. Moreover, this drive apparatus can also be retrofitted to existing lithotripsy devices.

In principle, a “drive apparatus” is any type of device that exerts a force upon the vibration excitation element and therefore causes a movement and/or rotation of the vibration excitation element.

In a further embodiment, the drive apparatus has a nozzle for effecting a compressed gas flow on the vibration excitation element.

This allows the vibration excitation element to be driven directly in a simple manner by means of a compressed gas flow—for example, compressed air. In this case, the drive and therefore the supply of the compressed gas flow can be continuous and/or discontinuous. The geometry of the nozzle and/or regulation of the compressed air flow can therefore be used to directly influence the movement frequency and pulse intensity of the vibration excitation element. Above all, the force, the torque, and/or the rotational speed of the vibration excitation element can be influenced quickly and directly by means of the compressed gas flow. In addition, the nozzle can be specifically aligned to and/or on the vibration excitation element, and the compressed gas flow can be applied to a defined position and/or location of the rotating vibration excitation element.

The term “nozzle” refers in particular to a technical component for influencing a fluid and/or compressed gas flow upon transitioning from a pipe flow into free space. The nozzle can have the same cross-sectional area over its entire length, widen, taper, and/or have a variable shape.

In order to drive the rotation of the vibration excitation element in a defined and positioned manner, the vibration excitation element has a groove in one lateral surface for effecting the compressed gas flow.

Preferably, the vibration excitation element has several grooves in its lateral surface that are evenly spaced from one another around the entire circumference of the lateral surface so that the flow of compressed gas flows into the particular groove which is located in the effective area of the nozzle, and, as is the case with an impeller, the vibration excitation element is driven to rotate.

The “lateral surface” of the vibration excitation element is in particular the surface on which the vibration excitation element can roll.

A “groove” is in particular an elongated depression in the vibration excitation element. One groove or several grooves are arranged radially circumferentially in particular on and/or in the lateral surface. Since the compressed gas flow is directed into the relevant groove by means of the nozzle and acts against the boundary wall at the end of the groove in the direction of flow, the vibration excitation element is set in rotation about the sonotrode.

In a further embodiment, the lithotripsy device has an attachable holder housing, wherein the attachable holder housing surrounds the vibration excitation element, a proximal end of the sonotrode, and/or the ultrasonic vibration exciter.

The surrounding holder housing makes it easier on the one hand to clean the lithotripsy device, and on the other prevents damage to the rotating vibration excitation element. In this case, the attachable holder housing can form part of the hand-held device and, just like the sonotrode, only partially surround the ultrasonic vibration generator.

To provide easy handling and cleanability, the material of the attachable holder housing can be of plastic, for example.

In order to provide a defined movement space for the rotation and wobbling of the vibration excitation element, the attachable holder housing has an internal proximal stop and/or a distal stop for limiting a movement path of the vibration excitation element along the longitudinal central axis of the sonotrode and/or a supply opening and an outlet opening for the compressed gas flow.

In addition to the distal stop and/or the proximal stop, the holder housing can also limit movement of the vibration excitation element in the transverse direction transverse to the longitudinal central axis of the sonotrode and/or all other spatial directions in between due to its shape and/or specifically arranged components.

A proximal stop and/or distal stop can be formed, for example, due to a shape on the inner surface of the attachable holder housing and/or due to an internal edge.

In a further embodiment, a spacer element is arranged between the proximal stop and the vibration excitation element and/or between the vibration excitation element and the distal stop.

The spacer element can therefore be used to specifically adjust and/or shorten the movement path of the vibration excitation element along the longitudinal central axis. Preferably, the spacer element is exchangeable, and different spacer elements with different lengths and/or masses can be used between the proximal stop and the vibration excitation element, and/or between the vibration excitation element and the distal stop.

The spacer element can be an elongated body and/or a spring, for example. Above all, with a distal arrangement, the spacer element can also be designed as an impact mass, which transmits a lateral pulse impact of the vibration excitation element and therefore transmits it in the axial direction in addition to the direct effect on the sonotrode.

In a further aspect of the invention, the object is achieved by a lithotripsy system for fragmenting body stones, wherein the lithotripsy system has an above-described lithotripsy device, a plurality of vibration excitation elements with a particular imbalance, and/or a plurality of sonotrodes.

This provides a lithotripsy system with which the user can quickly and easily adapt the lithotripsy device to the respective requirements of fragmentation by replacing a vibration excitation element with another vibration excitation element and/or a previously used sonotrode with another sonotrode, and achieve the optimum fragmentation performance in each case.

The exchange of vibration excitation elements with a particular imbalance and/or of sonotrodes with each other takes place in particular before a surgery, between a surgery, and/or outside the body in which a body stone is to be fragmented, or in a trial run.

In an additional aspect of the invention, the object is achieved by a retrofitting kit for retrofitting an existing lithotripsy device, wherein the existing lithotripsy device has a sonotrode and an ultrasonic vibration exciter, and the retrofitting kit has at least one vibration exciter element with an imbalance and with a through-opening for sliding onto the existing sonotrode, a drive apparatus for effecting a rotation of the vibration excitation element, and/or an attachable holder housing for surrounding the vibration excitation element, a proximal end of the sonotrode, and/or the ultrasonic vibration exciter such that a lithotripsy device as described above can be formed.

It is particularly advantageous that the retrofitting kit can also be used to retrofit sonotrodes and/or lithotripsy devices already on the market, in order to specifically excite a second vibration of the sonotrode. A drive apparatus can also be retrofitted to separately and specifically drive the vibration excitation element with the imbalance. For example, a nozzle for supplying compressed air can be fastened to the hand-held device and/or to the attachable holder housing.

In a further aspect, the object is achieved by a method for operating a lithotripsy device, wherein the lithotripsy device has an ultrasonic vibration exciter, a sonotrode with an outer diameter, a vibration excitation element with an imbalance and with a through-opening, and a drive apparatus for driving a rotation of the vibration excitation element, wherein the vibration excitation element is arranged with the through-opening around the outer diameter of the sonotrode, and the through-opening of the vibration excitation element has a larger diameter than the outer diameter of the sonotrode so that the vibration excitation element is freely movable, with the following steps:
Exciting a first vibration of the sonotrode by means of the ultrasonic vibration exciter,
Driving the rotation of the vibration excitation element around the sonotrode by means of the drive apparatus, and
Exciting a second vibration of the sonotrode by means of the imbalance of the rotating vibration excitation element.

In principle, these method steps can be carried out and/or repeated simultaneously or alternatingly in any order. If the method steps are carried out simultaneously, there is a combined operation and a dual effect by means of the first vibration and the second vibration.

In this way, very easily and independently of the ultrasonic vibration of the sonotrode, the user can impose a second vibration—in particular, a shock wave vibration—by driving the rotation of the vibration excitation element about the sonotrode. By using the two independent drive systems for the first vibration and the second vibration, each can be optimally adjusted separately in order to achieve the optimum fragmenting performance required in each case.

In principle, it should be emphasized that the claimed method steps concern the operation and generation of two different vibrations within a lithotripsy device, and the method for operating a lithotripsy device does not have a treatment step and therefore does not constitute a treatment method.

The invention is explained in more detail below with reference to exemplary embodiments. In the following:

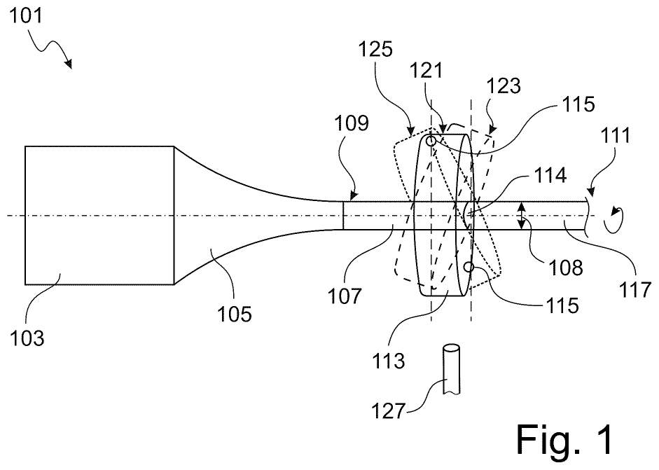

FIG. 1 shows a highly schematic representation of a lithotripsy device with an imbalance mass around the sonotrode in various vibration positions in longitudinal section,

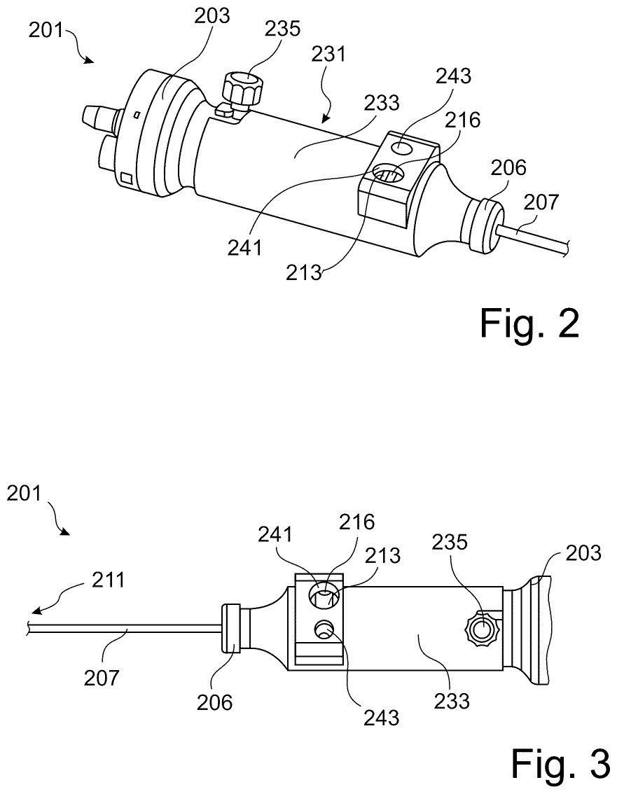

FIG. 2 shows a three-dimensional representation of an alternative lithotripsy device with a slip-on sleeve around an imbalance disk in a side view,

FIG. 3 shows a three-dimensional representation of the lithotripsy device from FIG. 2 in another side view,

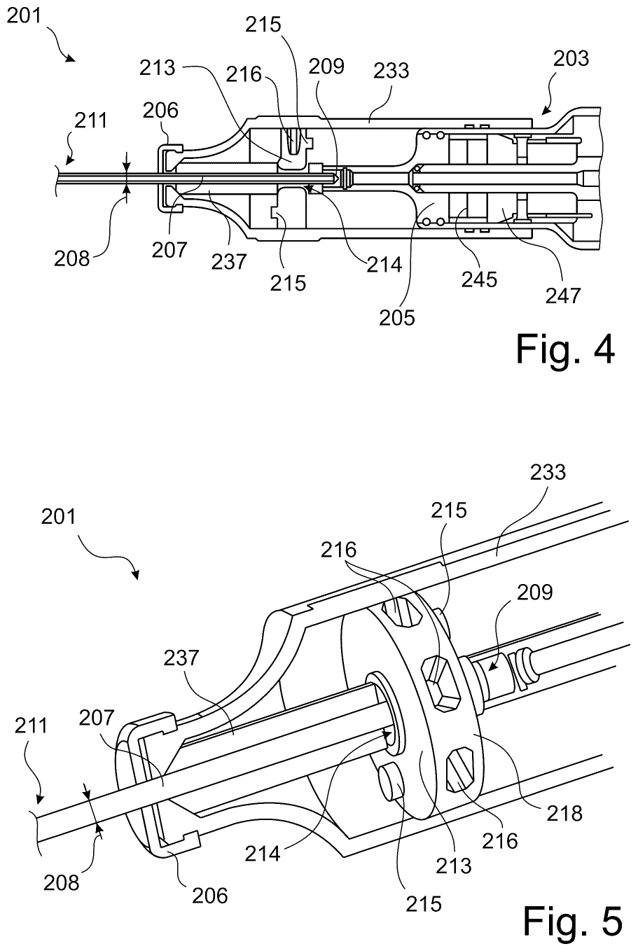

FIG. 4 shows a schematic representation of the lithotripsy device from FIG. 2 in longitudinal section,

FIG. 5 shows a three-dimensional, enlarged representation of a section of the lithotripsy device from FIG. 2 with the imbalance disk, and

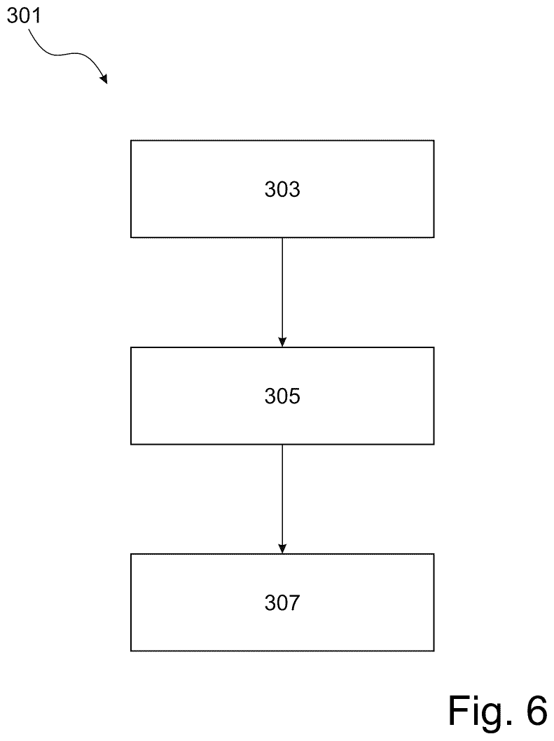

FIG. 6 shows a schematic representation of method steps of a method for operating a lithotripsy device.

A lithotripsy device 101 has an ultrasonic transducer 103 on the proximal side for generating an ultrasonic vibration. A tapered horn 105 is arranged on the distal side of the ultrasonic transducer 103. A sonotrode 107 is screwed into the horn 105 at its proximal end 109. An opposite distal end 111 of the sonotrode is used to fragment body stones. In the proximal region of the sonotrode 107, an imbalance mass 113 is pushed onto the sonotrode 107 by means of a central opening 114 and surrounds the sonotrode 107. Since the sonotrode 107 has a smaller outer diameter 108 than a diameter of the opening 114 of the imbalance mass 113, the imbalance mass 113 is freely movable around the sonotrode 107. The imbalance mass 113 is disk-shaped and has two overweights 115, wherein one overweight 115 is arranged proximally, and the other overweight 115 is arranged distally, but not rotationally symmetrically, on the imbalance mass 113.

A compressed air nozzle 127 for driving a rotation of the imbalance mass 113 about an axis of rotation 117 is arranged below the imbalance mass 113, wherein the axis of rotation 117 coincides with a longitudinal central axis of the sonotrode 107 and a longitudinal central axis of the lithotripsy device 101. The compressed air nozzle 127 is connected to a central compressed air supply (not shown) by means of a hose (not shown in FIG. 1). Here, the compressed air nozzle 127 and the hose (not shown) are held on the holder housing (not shown in FIG. 1) of the lithotripsy device 101.

To operate the lithotripsy device 101, the sonotrode 107 is continuously excited with an ultrasonic vibration by means of the ultrasonic transducer 103. Since the imbalance mass 113 is arranged around the sonotrode 107, this ultrasonic vibration also acts upon the imbalance mass 113, but leads only to a slight back-and-forth movement along the axis of rotation 117. The compressed air nozzle 127 is used to specifically apply compressed air to the imbalance mass 113 in the region of its lateral surface, whereby the imbalance mass 113 with its internal opening 114 rotates about the sonotrode 107 and the axis of rotation 117. Due to the two, non-rotationally symmetrically arranged overweights 115 on the proximal and distal sides of the imbalance mass 113, the imbalance mass 113 wobbles between a distal vibration position 123 and a proximal vibration position 125 in the rotating state, starting from an initial position 121 in a state not driven by the compressed air nozzle 127.

The imbalance mass 113 is made of stainless steel and accordingly strikes the sonotrode 107 hard alternately with its distal edge around the opening 114 and its proximal edge around the opening 114 while wobbling, wherein the sonotrode 107 is excited to a second vibration due to this impact excitation. Thus, the continuous, constant ultrasonic vibration excited by means of the ultrasonic transducer 103 and the intermittent shock vibration due to the shock excitation by means of the imbalance mass 113 with the two overweights 115 can be optimally used in a dual action mechanism to fragment body stones by means of the distal end 111 of the sonotrode 107. Thus, the sonotrode 107 is additionally excitable with a shock vibration functionally, temporally, and locally independent of an excitation of an ultrasonic vibration by means of the ultrasonic transducer 103, by using a dynamic imbalance of the imbalance mass 113 with a hard shock excitation.

In an alternative shown in FIGS. 2 to 5, a lithotripsy device 201 has an ultrasonic transducer 203 and a cap 206, wherein a hand-held device 231 with a slip-on sleeve 233 is arranged on the outside between the ultrasonic transducer 203 and the cap 206, wherein the slip-on sleeve 233 is attached to the ultrasonic transducer 203 by means of a lock 235. A sonotrode 207 is largely exposed up to its distal end 211 and is guided internally only through the cap 206 up to its proximal end 209 within the hand-held device 231 (FIG. 4).

In front of the proximal end 209 of the sonotrode 207, an imbalance disk 213 is arranged with several, evenly spaced grooves 216 along its lateral surface 218. In this case, a central opening 214 of the imbalance disk 213 surrounds the sonotrode 207. An overweight 215 is arranged on a distal side and a proximal side of the imbalance disk 213. A spacer sleeve 237 is arranged between the distal side of the imbalance disk 213 and the proximal inner side of the slip-on sleeve 233.

The sonotrode 207 in turn has a smaller outer diameter 208 than the central opening 214 of the imbalance disk 213 so that the imbalance disk 213 is freely movable around the sonotrode 207 within the slip-on sleeve 233. The proximal end 209 of the sonotrode 207 is mounted on the distal end of the horn 205. On the proximal side of the horn 205, the ultrasonic transducer 203 has a plurality of piezo elements 245 and a counter bearing 247.

On the outside, the slip-on sleeve 233 has an inlet opening 241 and an outlet opening 243 for compressed air. A nozzle, not shown in FIGS. 2 to 5, directs a stream of compressed air into the supply opening 241 onto the particular groove 216 for driving the imbalance disk 213.

The following operations are carried out with the lithotripsy device 201:

In a method 301 for operating the lithotripsy device 201, the piezo elements 245 are subjected to a voltage from an ultrasonic generator (not shown) (FIG. 6). Due to the resulting deformation of the piezo elements 245 which are clamped between the proximal counter bearing 247 and the distal horn 205, an ultrasonic vibration is induced, which results in an excitation 303 of a first vibration of the sonotrode 207. At the same time, a driving 305 of a rotation of the imbalance disk 213 about the sonotrode 207 is performed by directing a flow of compressed air into the groove 216 of the rotating imbalance disk 213, which is in the region of the feed opening 241, by means of the nozzle (not shown), whereby the imbalance disk 213 rotates at a continuous rotational speed. Due to the design of the imbalance disk 213 with the overweights 215, an excitation 307 of a second vibration of the sonotrode 207 occurs by means of a dynamic imbalance of the rotating imbalance disk 213.

Due to the separate adjustability of the efficiency of the excitation 303 of the first continuous ultrasonic vibration of the sonotrode 207 by means of the ultrasonic transducer 203, and of the excitation 307 of the second shock vibration of the sonotrode 207 by means of the rotating imbalance disk 213, two independent mechanisms of action can be optimally used to fragment even hard and/or inhomogeneous body stones.

The invention relates to a lithotripsy device for fragmenting body stones, wherein the lithotripsy device has a hand-held device with an ultrasonic vibration exciter with an ultrasonic vibration exciter outer diameter and a sonotrode with a longitudinal central axis and a sonotrode outer diameter which can be connected to the hand-held device on the distal side, wherein the sonotrode can be excited to a first vibration by means of the ultrasonic vibration exciter, and the lithotripsy device has a vibration excitation element with a through-opening, wherein the vibration excitation element with the through-opening is arranged around the sonotrode outer diameter and/or around the ultrasonic vibration exciter outer diameter, wherein the through-opening of the vibration excitation element has a larger diameter than the sonotrode outer diameter and/or than the ultrasonic vibration exciter outer diameter so that the vibration excitation element is freely movable, and the vibration excitation element has an imbalance so that, in the event of a rotation of the vibration excitation element about the sonotrode and/or the ultrasonic vibration exciter, the sonotrode can be excited to a second vibration by means of the imbalance. Furthermore, the invention relates to a lithotripsy system, a retrofitting kit for retrofitting an existing lithotripsy device, and a method for operating a lithotripsy device.

### LIST OF REFERENCE SIGNS

101 Lithotripsy device
103 Ultrasonic transducer
105 Horn
107 Sonotrode
108 Outer diameter of the sonotrode
109 Proximal end of the sonotrode
111 Distal end of the sonotrode
113 Imbalance mass
114 Opening
115 Overweight
117 Axis of rotation/longitudinal central axis of the sonotrode
121 Initial position
123 Distal vibration position
125 Proximal vibration position
127 Compressed air nozzle
201 Lithotripsy device
203 Ultrasonic transducer
205 Horn
206 Cap
207 Sonotrode
208 Outer diameter of the sonotrode
209 Proximal end of the sonotrode
211 Distal end of the sonotrode
213 Imbalance disk
214 Opening
215 Overweight
216 Groove
218 Lateral surface
231 Hand-held device
233 Slip-on sleeve
235 Lock
237 Spacer sleeve
241 Inlet opening for compressed air
243 Outlet opening for compressed air
245 Piezo elements
247 Counter bearing
301 Method for operating a lithotripsy device
303 Excitation of a first vibration of a sonotrode
305 Driving a rotation of an imbalance mass about the sonotrode
307 Exciting a second vibration of the sonotrode by means of an imbalance of the rotating imbalance mass

1. Lithotripsy device (101, 201) for fragmenting body stones, wherein the lithotripsy device (101, 201) has a hand-held device (231) with an ultrasonic vibration exciter (103, 203) with an ultrasonic vibration exciter outer diameter and a sonotrode (107, 207) with a longitudinal central axis (117) and a sonotrode outer diameter (108, 208), which can be connected to the hand-held device (231) on the distal side, wherein the sonotrode (107, 207) can be excited to a first vibration by means of the ultrasonic vibration exciter (103, 203), and the lithotripsy device (101, 201) has a vibration excitation element (113, 213) with a through-opening (114, 214), wherein the vibration excitation element (113, 213) with the through-opening (114, 214) is arranged around the sonotrode outer diameter (108, 208) and/or around the ultrasonic vibration exciter outer diameter, characterized in that the through-opening (114, 214) of the vibration excitation element (113, 213) has a larger diameter than the sonotrode outer diameter (108, 208) and/or than the ultrasonic vibration exciter outer diameter so that the vibration excitation element (113, 213) is freely movable, and the vibration excitation element (113, 213) has an imbalance so that, in the event of a rotation of the vibration excitation element (113, 213) about the sonotrode (107, 207) and/or the ultrasonic vibration exciter, the sonotrode (107, 207) can be excited to a second vibration by means of the imbalance.

2. Lithotripsy device (101, 201) according to claim 1, characterized in that the vibration excitation element (113, 213) is disc-shaped, annular, hollow-cylinder-shaped, and/or toroidal.

3. Lithotripsy device (101, 201) according to claim 1 or 2, characterized in that the vibration excitation element (113, 213) has an axis of rotation deviating from the longitudinal central axis (117) and/or its main axis of inertia.

4. Lithotripsy device (101, 201) according to any of the preceding claims, characterized in that the vibration excitation element (113, 213) has an imbalance element (115, 215) in its interior and/or on its outer surface.

5. Lithotripsy device (101, 201) according to any of the preceding claims, characterized in that the vibration excitation element (113, 213) has a recess.

6. Lithotripsy device (101, 201) according to claim 4 or 5, characterized in that the imbalance element (115, 215) and/or the recess is or are arranged on a distal side and/or a proximal side of the vibration excitation element (113, 213).

7. Lithotripsy device (101, 201) according to any of the preceding claims, characterized in that the lithotripsy device (101, 201) has a drive apparatus for driving the rotation of the vibration excitation element (113, 213).

8. Lithotripsy device (101, 201) according to claim 7, characterized in that the drive apparatus has a nozzle (127) for effecting a compressed gas flow on the vibration excitation element (113, 213).

9. Lithotripsy device (101, 201) according to claim 8, characterized in that the vibration excitation element (113, 213) has a groove (216) in a lateral surface (218) for effecting the compressed gas flow.

10. Lithotripsy device (101, 201) according to any of the preceding claims, characterized in that the lithotripsy device (101, 201) has an attachable holder housing (233), wherein the attachable holder housing (233) surrounds the vibration excitation element (113, 213), a proximal end (109, 209) of the sonotrode (107, 207) and/or the ultrasonic vibration exciter (103, 203).

11. Lithotripsy device (101, 201) according to claim 10, characterized in that the attachable holder housing (233) internally has a proximal stop and/or a distal stop for limiting a movement path of the vibration excitation element along the longitudinal central axis (117) of the sonotrode and/or an inlet opening (241) and an outlet opening (243) for the compressed gas flow.

12. Lithotripsy device (101, 201) according to claim 11, characterized in that a spacer element (237) is arranged between the proximal stop and the vibration excitation element (113, 213) and/or between the vibration excitation element (113, 213) and the distal stop.

13. Lithotripsy system for fragmenting body stones, characterized in that the lithotripsy system has a lithotripsy device (101, 201) according to any of claims 1 to 12, a plurality of vibration excitation elements with a relevant imbalance (113, 213), and/or a plurality of sonotrodes (107, 207).

14. Retrofitting kit for retrofitting an existing lithotripsy device, wherein the existing lithotripsy device has a sonotrode (107, 207) and an ultrasonic vibration exciter (103, 203), characterized in that the retrofitting kit has at least one vibration excitation element (113, 213) with an imbalance and with a through-opening (114, 214) for sliding onto the existing sonotrode (107, 207), a drive apparatus for effecting a rotation of the vibration excitation element (113, 213), and/or an attachable holder housing (233) for surrounding the vibration excitation element (113, 213), a proximal end (109, 209) of the sonotrode (107, 207) and/or the ultrasonic vibration exciter (103, 203), such that a lithotripsy device (101, 201) according to any of claims 1 to 12 can be formed.

15. Method (301) for operating a lithotripsy device (101, 201), wherein the lithotripsy device (101, 201) has an ultrasonic vibration exciter (103, 203), a sonotrode (107, 207) with an outer diameter (108, 208), a vibration excitation element (113, 213) with an imbalance and with a through-opening (114, 214), and a drive apparatus for driving a rotation of the vibration excitation element (113, 213), wherein the vibration excitation element (113, 213) is arranged with the through-opening (114, 214) around the outer diameter (108, 208) of the sonotrode (107, 207), and the through-opening (114, 214) of the vibration excitation element (113, 213) has a larger diameter than the outer diameter (108, 208) of the sonotrode (107, 207) so that the vibration excitation element (113, 213) is freely movable, comprising the following steps:
Exciting (303) a first vibration of the sonotrode (107, 207) by means of the ultrasonic vibration exciter (103, 203),
Driving (305) the rotation of the vibration excitation element (113, 213) about the sonotrode (107, 207) by means of the drive apparatus, and
Exciting (307) a second vibration of the sonotrode (107, 207) by means of the imbalance of the rotating vibration excitation element (113, 213).
